(19) United States
(12) Plant Patent Application Publication
(10) Pub. No.: US 2018/0279531 P1
(43) Pub. Date: Sep. 27, 2018

(54) Bidens plant named 'Angel Rouge'

(50) ;

(71) Applicant: Tsunotan IP Co., Ltd., Hagiwara-cho (JP)

(72) Inventor: Koichiro Nishikawa, Naghi-cho (JP)

(21) Appl. No.: 15/932,616

(22) Filed: Mar. 23, 2018

(30) Foreign Application Priority Data
Mar. 24, 2017 (JP) 31958

(51) Int. Cl. A01H 6/14

(52) U.S. Cl. PLT/410

(57) ABSTRACT

A new and distinct bidens plant having dark, orange-red colored ray florets and a compact growth habit.

Botanical classification: Bidens L.

Varietal denomination: ‘Angel Rouge’.

### CROSS REFERENCE TO RELATED APPLICATION(S)

This application claims the benefit of priority from Japanese Plant Variety Protection Application No. 31958, filed Mar. 24, 2017, which is incorporated herein by reference in its entirety.

### BACKGROUND OF THE INVENTION

The present invention comprises a new and distinct bidens plant known by the varietal name ‘Angel Rouge’. The new variety is the result of a planned breeding program with the purposes of providing bidens plants with a red flower color and a compact growth habit. The breeding process leading up to ‘Angel Rouge’ began in 2012 from the applicant's proprietary collection of bidens plants. Ultimately, ‘Angel Rouge’ is the result of a 2014 cross between Bidens L. varieties referred to as BR5-3 (female parent, unpatented) and Nunca Y-1 (male parent, unpatented) and was discovered in 2015 in Aichi Prefecture, Japan. The first asexual reproduction of the new variety was conducted by cuttings in 2016 in Aichi Prefecture, Japan. ‘Angel Rouge’ has been trial and field tested and has been found to retain its distinctive characteristics and remain true to type through successive propagations. The present invention has not been evaluated under all possible environmental conditions. The phenotype may vary with variations in environment without a change in the genotype of the plant.

When ‘Angel Rouge’ is compared to Bidens L. ‘Angel Cheek’ (unpatented), both varieties exhibit a compact growth habit and orange-red colored ray florets. However, the ray florets of ‘Angel Rouge’ have a solid and dark, orange-red colored upper surface, while the ray florets of ‘Angel Cheek’ have a lighter, primary orange-red color and a secondary orange-yellow color present at the upper surface base and tip. Further, ‘Angel Rouge’ exhibits no to less anthocyanin stem coloration when compared to ‘Angel Cheek’. Additionally, ‘Angel Rouge’ has an earlier time of flowering than ‘Angel Cheek’.

The following traits distinguish ‘Angel Rouge’ as a new and distinct cultivar from other bidens varieties known to the breeder:
1. Compact growth habit;
2. Short plant height;
3. Absent or very weak anthocyanin stem coloration;
4. Pinnate leaves;
5. Medium-sized leaves;
6. No leaf variegation present;
7. Single inflorescences (less than 8 ray florets);
8. Medium inflorescence diameter;
9. Medium ray floret length and width;
10. Shallow ray floret apex incisions;
11. Solid and dark, orange-red colored upper surface of ray florets;
12. Early time of flowering; and
13. Continuous flowering habit.

### DESCRIPTION OF THE DRAWINGS

The accompanying photographic images captured on Mar. 7, 2017 illustrate the new variety, with the colors being as nearly true as is possible with color illustrations of this type:

FIG. 1 illustrates an entire flowering plant of the new variety;

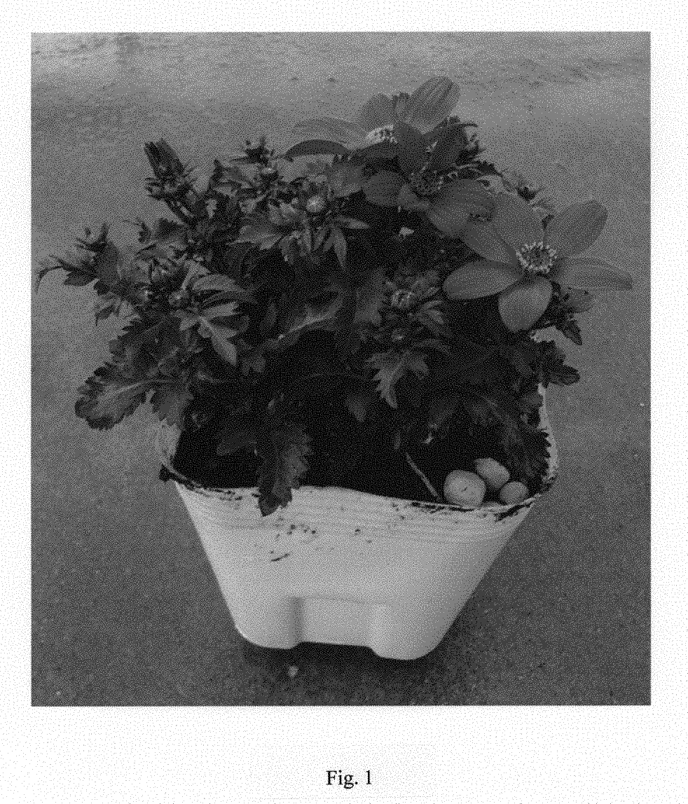

FIG. 2 illustrates numerous flowering plants of the new variety;

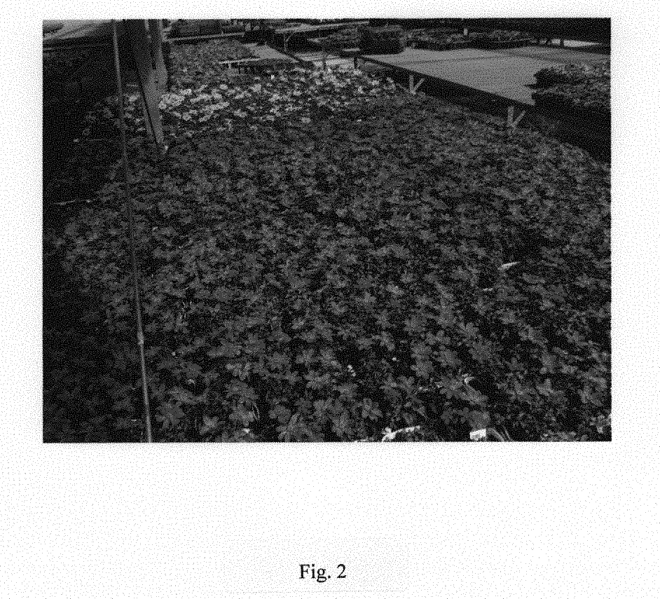

FIG. 3 illustrates a flowering plant of ‘Angel Rouge’ (left hand side) and its comparison variety, ‘Angel Cheek’ (right hand side);

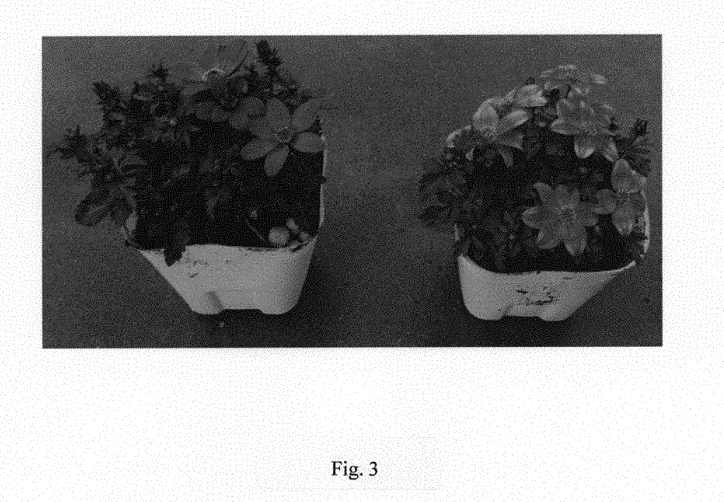

FIG. 4 illustrates the upper surface of an inflorescence of the new variety; and

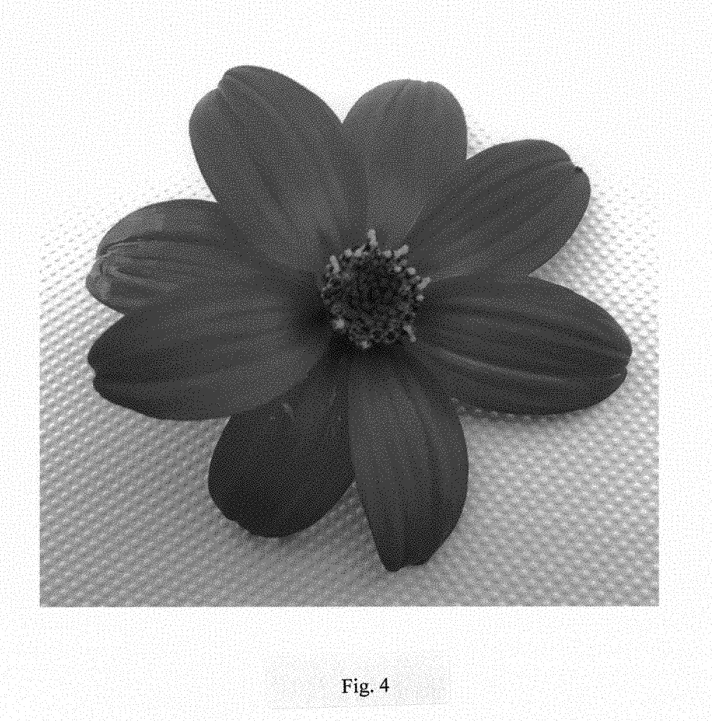

FIG. 5 illustrates the lower surface of an inflorescence of the new variety.

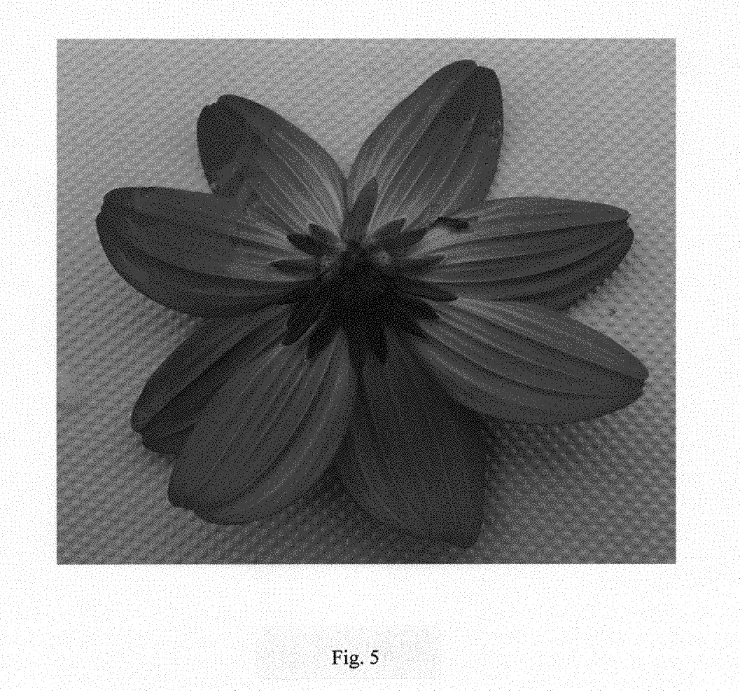

### DESCRIPTION OF THE PLANT

The following detailed description sets forth the characteristics of the new variety. The data which defines these characteristics was collected in Aichi Prefecture, Japan under natural light.
Plant:
   Intended use.—For planting and gardening purposes.
   Optimal planting/cutting season.—From the beginning of December to the beginning of January and from late August to the beginning of November in Aichi Prefecture, Japan.
   Overall plant shape.—Compact and upright.
   Plant height.—Short.
   Stem anthocyanin coloration.—Absent or very weak.
   Leaves.—Type: Pinnate. Size: Medium. Color: Dark green. Variegation: Absent.
Inflorescence:
   Natural flowering season.—From late October to late June in Aichi Prefecture, Japan. Time of flowering: Early. Type and habit: Single and continuous. Head diameter: Medium. Inflorescence position: Above foliage.
   Ray florets.—Length: Medium. Width: Medium. Shape: Oval. Apex: Rounded with shallow incisions. Margin: Entire. Number per inflorescence: 8. Color: Upper surface: Solid and dark, orange-red. Lower surface: Dark, orange-red with striations of yellow-orange.

I claim:

1. A new and distinct variety of bidens plant, as is herein illustrated and described.
